(19) United States
(12) Patent Application Publication
(10) Pub. No.: US 2026/0247088 A1
(43) Pub. Date: Aug. 20, 2026

(54) HEARING AID FOR ADJUSTING A PROCESSING PARAMETER

(71) Applicant: Oticon A/S, Smørum (DK)

(72) Inventors: Jeppe Høy CHRISTENSEN, Smørum (DK); Jesper JENSEN, Smørum (DK); Michael Syskind PEDERSEN, Smørum (DK)

(21) Appl. No.: 19/533,332

(22) Filed: Feb. 9, 2026

(30) Foreign Application Priority Data
Feb. 17, 2025 (EP) 25158265.6

(51) Int. Cl. H04R 25/00

(52) U.S. Cl. CPC H04R 25/70; H04R 25/305; H04R 25/552; H04R 25/554; H04R 2225/43

(57) ABSTRACT

Disclosed herein are embodiments of a hearing aid configured for being worn by a user. The hearing aid can include a processor configured to: obtain a hearing aid audio signal based on the input audio signal, determine a first audio parameter based on the input audio signal, obtain a first sensor signal indicative of a first biological parameter of the user, determine the first biological parameter based on the first sensor signal, determine a first correlation between the first audio parameter and the first biological parameter, and set a processing parameter of the hearing aid based on the first correlation.

### CROSS REFERENCE TO RELATED APPLICATIONS

Any and all applications for which a foreign or domestic priority claim is identified in the Application Data Sheet as filed with the present application are hereby incorporated by reference under 37 CFR 1.57.

### TECHNICAL FIELD

The present application relates to the field of hearing aids.

### BACKGROUND

Individuals with a hearing impairment have difficulty listening to sounds in noisy and loud environments. Hearing aids with noise reduction algorithms can help suppress unwanted noise and improve the perceived signal-to-noise ratio of the user.

In addition, noise reduction algorithms can set the level of noise suppression depending on the ambient signal-to-noise ratio and sound pressure levels (e.g., Oticon Open Sound Navigator™). However, doing so neglects the state of the user. That is, is the user in true need of more aggressive noise reduction, or not? While the environment might be noisy and loud, this alone cannot tell if an end user would like the hearing aid to increase noise reduction.

Research shows that human heart rate (HR) is sensitive to changes in listening conditions defined by the characteristics of the ambient sound. Especially, increases in ambient sound intensity (sound pressure levels) translate to linear increases in HR, while increases in sound clarity (signal-to-noise ratio) correspond to decreases in HR. The latter effect is even more pronounced in overall louder environments with sound pressure levels higher than the most typical observed for hearing aid users (60 dB).

The sensitivity of the HR to ambient sound (e.g., the change in beats-per-minute for each change in decibel SPL or SNR) may be considered a stress response and results from both direct biological mechanisms and indirectly from cognitive mechanisms.

### SUMMARY

In an aspect of the present application, a hearing aid is provided. The hearing aid may comprise an input unit for providing an input audio signal indicative of a surrounding of the hearing aid. The hearing aid may comprise a processor. The processor may be configured to obtain a hearing aid audio signal based on the input audio signal, determine a first audio parameter based on the input audio signal, obtain a first sensor signal indicative of a first biological parameter of the user, determine the first biological parameter based on the first sensor signal, determine a first correlation between the first audio parameter and the first biological parameter, and set a processing parameter of the hearing aid based on the first correlation.

Thereby an improved hearing aid may be provided. By monitoring changes in one or more biological parameters of the user resulting from changes in ambient sound, it may be learned if the current sound exposure causes stress or other discomfort. Information about correlations between the sound environment and the user's reaction to the sound can be fed to the processor of the hearing aid to help set processing to better suit the individual. Thus, a hearing aid according to the present disclosure may set processing parameters based on the biological parameters of the hearing aid user.

The first audio parameter may be a signal to noise ratio (SNR) determined based on the input audio signal. The first audio parameter may be a sound pressure level (SPL) determined based on the input audio signal. The first audio parameter may be a variation in SNR over time. The first audio parameter may be a variation in SPL over time. The first sound parameter may be a sound modulation level (SML). The first audio parameter may be an accumulated noise measure. The accumulated noise measure may be a measure for the time which a user been exposed to noise over a time period. The measure for when a user has been exposed to noise may be determined by setting a threshold defining when the user is exposed to noise. The time period may be an hour, two hours, a day, a week, or a month. The first audio parameter may be a determination of whether the user is listening to speech or noise. The determination of whether the user is listening to speech or noise may be conducted by a voice activity detector (VAD) implemented in the hearing aid.

The first biological parameter may be a heart rate (HR) of the user. The first biological parameter may be a pulse of the user. The first biological parameter may be a temperature of the user. The first biological parameter may be a sweat measurement of the user. The first biological parameter may be an electrodermal activity measurement. The first biological parameter may be an electroencephalogram (EEG) of the user. The first biological parameter may be a photoplethysmography (PPG) of the user. The first biological parameter may be a respiration measurement of the user. The first biological parameter may be an own voice of the user. The first biological parameter may be one or more voice characteristics of the user. The first biological parameter may be a pupil dilation of the user. The first biological parameter may be a fatigue measurement of the user.

The first biological parameter may be estimated based on a plurality of first sensor signals. The first biological parameter may be estimated as a joint parameter based on a plurality of first sensor signals. The plurality of first sensor signals may provide a level of redundancy, so as not to overly rely on the signal from a single sensor. The plurality of first sensor signals may be provided by a plurality of first sensors comprised by the hearing aid. The plurality of first sensor signals may be provided by a plurality of first sensors external to the hearing aid. The plurality of first sensor signals may be provided by a plurality of first sensors external to and comprised by the hearing aid. For example, if the hearing aid is part of a binaural hearing aid system one or more first sensor signals may be received by a processor of either of the hearing aid from each hearing aid of the binaural hearing aid system, thus, allowing the processor to estimate a joint parameter based on first sensor signals from each hearing aid.

The first biological parameter may be measured by an appropriate sensor comprised by the hearing aid or an external device communicatively connected to the hearing aid. The external device may be a smart device comprising one or more biological sensors. The external device may be paired with the hearing aid.

The first correlation may be a mutual relation or connection between the first audio parameter and the first biological parameter. The first correlation may define how the first audio parameter relates to the first biological parameter. The first correlation may be a mathematical correlation. The first correlation may be determined as the covariance of the first biological parameter and the first audio parameter divided by the product of their standard deviations, this may be expressed as:

$${ρ}_{BP , AP 1} = \frac{E [ BP 1 * AP 1 ] - E [ BP 1 ] * E [ AP 1 ]}{\sqrt{E [ BP {1}^{2} ] - {( E [ BP 1 ] )}^{2}} * \sqrt{E [ AP {1}^{2} ] - {( E [ AP 1 ] )}^{2}}}$$

Where BP1 denotes the first biological parameter, AP1 denotes the first audio parameter, ρ_{BP,AP} denotes the correlation between the first audio parameter and the first biological parameter, and the expected value E represents the mean.

The processing parameter set by the processor may be related to a hearing loss compensation algorithm, such as amplification or compression. The processing parameter set by the processor may be related to a noise reduction algorithm, such as a beamformer, filter coefficients, or update rates. The processing parameter set by the processor may be related to a feedback reduction algorithm, such as a feedback path estimation.

In the present disclosure setting a parameter may be understood as fixing a value of a parameter, changing a value of the parameter to a new value, or maintaining a value of the parameter.

In an embodiment the hearing aid comprises a first biological sensor configured to determine the first sensor signal.

The first biological sensor may be a PPG sensor. The first biological sensor may be a heart rate sensor. The first biological sensor may be arranged within a housing of the hearing aid. The biological sensor may be arranged externally to the housing of the hearing aid. The first biological sensor may be defined by one or more microphones comprised by the hearing aid. The first biological sensor may be a camera. The first biological sensor may be one or more electrodes configured to measure an EEG of the user. The first biological sensor may be an optical sensor. The first biological sensor may be an accelerometer configured to monitor a heart rate of the user.

In an embodiment the first biological sensor comprises an inward facing microphone.

By using a microphone as the first biological sensor it may obviate the need to add additional sensors to the hearing aid as the microphones already comprised by the hearing aid may function as the biological sensor.

By an inward facing microphone may be understood a microphone where the inlet of the microphone faces towards the ear, ear canal or ear drum of the user while the hearing aid is being worn by the user.

In an embodiment the first correlation is determined continuously over a plurality of time-windows.

The first correlation may be determined continuously across overlapping time-windows. The time-windows may vary in duration. An example of how a correlation may be determined continuously is provided by Christensen, Jeppe H., et al. “The everyday acoustic environment and its association with human heart rate: evidence from real-world data logging with hearing aids and wearables.” Royal Society open science 8.2 (2021): 201345. The time-windows may have a duration of 30 seconds, 1 min, 5 min, 1 hour, 2 hours, or a day.

In an embodiment the first biological parameter comprises a heart rate of the user.

In an embodiment the first audio parameter comprises a sound pressure level.

In an embodiment the processor is configured to set an aggressiveness of a noise reduction algorithm based on the first correlation.

The aggressiveness of the noise reduction algorithm may be viewed as a risk willingness to reduce noise versus the risk of introducing processing artefacts, e.g., if the noise reduction algorithm is set to a high aggressiveness it may indicate a low aversion to introducing processing artefacts while a low aggressiveness may indicate a high aversion to introducing processing artefacts. The aggressiveness may be given as an update rate of the noise reduction algorithm, e.g., an update rate of one or more adaptive filters of the noise reduction algorithm 20 and/or an update rate of a beamformer. A high update rate may indicate a high aggressiveness, and a low update rate may indicate a low aggressiveness. The aggressiveness may be given as a smoothing parameter of the noise reduction algorithm. The aggressiveness may be given as a maximum allowable gain change introduced by the noise reduction algorithm. A high maximum allowable gain change may indicate a high aggressiveness, and a low maximum allowable gain change may indicate a low aggressiveness. The aggressiveness may be given as an SNR ratio at which a noise reduction algorithm is enabled. A low SNR ratio for enabling the noise reduction algorithm may indicate a high aggressiveness, and a high SNR ratio for enabling the noise reduction algorithm may indicate a low aggressiveness.

In an embodiment the processor is configured to compare the first correlation to a first threshold range, and if the first correlation exceeds the first threshold range, increase an aggressiveness of a noise reduction algorithm of the hearing aid.

The first threshold range may be a personalized range set during fitting of the hearing aid. The first threshold range may be a preset range set during manufacturing of the hearing aid.

In an embodiment the processor is configured to determine a first estimate of causality between the first audio parameter and the first biological parameter, and set a processing parameter of the hearing aid based on the first estimate of causality.

By estimating a causality between the first audio parameter and the first biological parameter it gives a higher amount confidence on the foundation for setting a processing parameter. For example, if a high correlation is measured between a biological parameter and an audio parameter it may indicate a user responding biologically to changes in the sound environment, however, this could also be coincidence, by estimating the strength of a causality estimate it may give further information on whether the correlation was coincidental or a result of causal relationship.

Causality may be estimated by structural causal model (SCM), Rubin causal model or similar methods.

In an embodiment the first estimate of causality is a measure of temporal Granger causality.

An example of how Granger causality is estimated in relation to biological parameters may be found in Porta, Alberto, et al. “Cardiovascular control and time domain Granger causality: insights from selective autonomic blockade.” Philosophical Transactions of the Royal Society A: Mathematical, Physical and Engineering Sciences 371.1997 (2013): 20120161. Another example of using Granger causality may be found in Christensen, J., Andersson, K., & Neher, T. (2023 February) Distinct influence of everyday noise on cardiovascular stress In INTER-NOISE and NOISE-CON Congress and Conference Proceedings (Vol. 265, No. 7, pp. 242-247) Institute of Noise Control Engineering.

In an embodiment the processor is configured to obtain a second sensor signal indicative of an activity level of the user, determine the activity level based on the second sensor signal, and set a processing parameter of the hearing aid based on the activity level.

By including the activity level into a decision process for setting the processing parameter it may provide further information on why a change in a biological parameter is observed instead of purely relying on audio parameters, e.g., the activity level may indicate the user is running which is what causing an elevated heart rate.

The activity level may be a pace of the user. The activity level may be a determined distance moved by the user over a time period. The activity level may be a gait analysis.

In an embodiment the hearing aid comprises a second sensor configured to determine the second sensor signal.

The second sensor may comprise an inertial movement unit (IMU). The second sensor may comprise a gyroscope. The second sensor may comprise a global positioning system (GPS). The second sensor may comprise an accelerometer. An example of a hearing aid with a second sensor capable of tracking movement may for example be found in the Oticon Intent hearing aid.

In an embodiment the processor is configured to, over a period of time, store a plurality of first biological parameters, determine a user characteristic based on the plurality of first biological parameter, and set the processing parameter based on the user characteristic.

Thus, further information is provided to the processor in deciding whether to set the processing parameter to a new value or to maintain the current processing parameter. For example, a high correlation between the first biological parameter and the first audio parameter may indicate a stress reaction which could indicate a need to change the processing parameter, however, if the user characteristic is still within a normal range for the user no change in processing parameter may be needed yet, and only when the first biological parameter is outside the normal range for the user may a change in processing parameter be needed.

The first user characteristic may be a mean for the first biological parameter measured over the time period. The first user characteristic may be a range, where the range is defined by the mean and standard deviation of the first biological parameter, e.g., formulated as [E[BP1]−σ_{BP1}; E[BP1]+σ_{BP1}]. The first user characteristic may be a resting heart rate of the user.

In an embodiment the processor is configured to determine a second audio parameter based on the input audio signal, determine a second correlation between the second audio parameter and the first biological parameter, and set a processing parameter of the hearing aid based on the second correlation.

A further audio parameter may increase the confidence in setting a processing parameter of the hearing aid, and how to set the parameter.

In an embodiment the second audio parameter comprises a signal to noise ratio.

In an embodiment the processor is configured to compare the first correlation to a first threshold range, compare the second correlation to a second threshold range, and if the first correlation is within the first threshold range or falls short of the first threshold range, and the second correlation is within the second threshold range or falls short of the second threshold range, decrease an aggressiveness of a noise reduction algorithm of the hearing aid.

The second threshold range may be a personalized range set during fitting of the hearing aid. The second threshold range may be a preset range set during manufacturing of the hearing aid.

In an embodiment the processor is configured to compare the first correlation to a first threshold range, compare the second correlation to a second threshold range, and if the first correlation is within the first threshold range or falls short of the first threshold range, and the second correlation is within the second threshold range or exceeds the second threshold range except if both the first correlation and the second correlation is within the first threshold range and the second threshold range, maintain the processing parameter.

Table 1 below shows how different correlations may indicate different user states and how the hearing aid may intervene based on the different determined correlations. On the table below ρ_{HR,SPL} denotes a first correlation measure between HR and SPL, ρ_{HR,SNR} denotes a second correlation measure between HR and SNR, High+ denotes a positive correlation measure above a threshold range, 0 denotes a correlation measure within the threshold range, and High-denotes a negative correlation measure below the threshold range. A first threshold range may be associated with ρ_{HR,SPL}, and a second threshold range may be associated with ρ_{HR,SNR}.

TABLE 1
Potential outcomes and corresponding intervention.

| | | | Potential |
|---|---|---|---|
| ρ_{HR, SPL} | ρ_{HR, SNR} | Indication | intervention |
| High+ | 0 | Increasing SPL increases | Increase noise |
| | | HR - Stress reaction from | reduction |
| | | higher SPL | |
| High+ | High+ | Increasing SPL and SNR | Increase noise |
| | | increases HR - Stress | reduction |
| | | reaction from higher SPL | |
| | | and SNR | |
| High+ | High− | Increasing SPL increases | Increase noise |
| | | HR but increasing SNR | reduction but not |
| | | decrease HR - Stress | amplification |
| | | reaction from higher SPL | |
| | | but decrease in stress from | |
| | | SNR | |
| High− | 0 | Increasing SPL decreases | No intervention |
| | | HR - No stress reaction | |
| High− | High+ | Increasing SPL decreases | No intervention |
| | | HR but increasing SNR | needed. |
| | | increase HR - Stress | |
| | | reaction from speech-like | |
| | | sounds | |
| High− | High− | Increasing SPL and SNR | Decrease noise |
| | | decreases HR - De-stress | reduction. |
| | | reaction | |
| 0 | 0 | No impact of SPL or SNR | Decrease noise |
| | | on HR - No stress reaction | reduction. |
| 0 | High+ | Increasing SNR increase | No intervention |
| | | HR - Stress reaction from | needed. |
| | | speech-like sounds. | |
| 0 | High− | Increasing SNR decreases | Decrease noise |
| | | HR - De-stress reaction | reduction. |
| | | from speech-like sounds. | |

The hearing aid may be adapted to provide a frequency dependent gain and/or a level dependent compression and/or a transposition (with or without frequency compression) of one or more frequency ranges to one or more other frequency ranges, e.g. to compensate for a hearing impairment of a user. The hearing aid may comprise a signal processor for enhancing the input signals and providing a processed output signal.

The hearing aid may comprise an output unit for providing a stimulus perceived by the user as an acoustic signal based on a processed electric signal. The output unit may comprise an output transducer. The output transducer may comprise a receiver (loudspeaker) for providing the stimulus as an acoustic signal to the user. The output transducer may comprise a vibrator for providing the stimulus as mechanical vibration of a skull bone to the user. The output unit may comprise a transmitter for transmitting sound picked up-by the hearing aid to another device, e.g. a far-end communication partner.

The hearing aid may comprise an input unit for providing an input audio signal representing sound. The input unit may comprise an input transducer, e.g., a microphone, for converting an input sound to an input audio signal. The input unit may comprise a wireless receiver for receiving a wireless signal comprising or representing sound and for providing an input audio signal representing said sound.

The wireless receiver and/or transmitter may, e.g., be configured to receive and/or transmit an electromagnetic signal in the radio frequency range (3 kHz to 300 GHz). The wireless receiver and/or transmitter may, e.g., be configured to receive and/or transmit an electromagnetic signal in a frequency range of light (e.g., infrared light 300 GHz to 430 THz, or visible light, e.g., 430 THz to 770 THz).

The hearing aid may comprise a directional microphone system adapted to spatially filter sounds from the environment and thereby enhance a target acoustic source among a multitude of acoustic sources in the local environment of the user wearing the hearing aid. The directional system may be adapted to detect from which direction a particular part of the microphone signal originates. This can be achieved in various different ways, e.g. described in the prior art. In hearing aids, a microphone array beamformer is often used for spatially attenuating background noise sources. The beamformer may comprise a linear constraint minimum variance (LCMV) beamformer. Many beamformer variants can be found in literature. The minimum variance distortionless response (MVDR) beamformer is widely used in microphone array signal processing. Ideally the MVDR beamformer keeps the signals from the target direction (also referred to as the look direction) unchanged, while attenuating sound signals from other directions maximally. The generalized sidelobe canceller (GSC) structure is an equivalent representation of the MVDR beamformer offering computational and numerical advantages over a direct implementation in its original form.

The hearing aid may comprise antenna and transceiver circuitry allowing a wireless link to an entertainment device, a communication device, a wireless microphone, an external processing device, or another hearing aid, etc. The hearing aid may thus be configured to wirelessly receive a direct input signal from another device. Likewise, the hearing aid may be configured to wirelessly transmit an output signal to another device. The input or output signal may represent or comprise an audio signal and/or a control signal and/or an information signal.

In general, a wireless link established by antenna and transceiver circuitry of the hearing aid can be of any type. The wireless link may be a link based on near-field communication, e.g., an inductive link based on an inductive coupling between antenna coils of transmitter and receiver parts. The wireless link may be based on far-field, electromagnetic radiation. Preferably, frequencies used to establish a communication link between the hearing aid and the other device is below 70 GHz, e.g. located in a range from 50 MHz to 70 GHz, e.g. above 300 MHz, e.g. in an ISM range above 300 MHz, e.g. in the 900 MHz range or in the 2.4 GHz range or in the 5.8 GHz range or in the 60 GHz range (ISM=Industrial, Scientific and Medical, such standardized ranges being e.g. defined by the International Telecommunication Union, ITU). The wireless link may be based on a standardized or proprietary technology. The wireless link may be based on Bluetooth technology (e.g. Bluetooth Low-Energy technology, e.g. LE audio), or Ultra WideBand (UWB) technology.

The hearing aid may comprise a signal path for processing an audio signal. A signal processor may be adapted to provide a frequency dependent gain according to a user's particular needs. Some or all signal processing may be conducted in the frequency domain, in which case the hearing aid comprises appropriate analysis and synthesis filter banks. Some or all signal processing may be conducted in the time domain.

The hearing aid may comprise a number of sensors configured to provide status signals relating to a current physical environment of the hearing aid, and/or to a current state of the user wearing the hearing aid, and/or to a current state or mode of operation of the hearing aid. Alternatively, or additionally, one or more sensors may form part of an external device in communication with the hearing aid. An external device may e.g. comprise another hearing aid, a remote control, and audio delivery device, a telephone, an external sensor, etc.

The hearing aid may comprise a voice activity detector (VAD) for estimating whether or not an input signal comprises a voice signal. A voice signal may in the present context be taken to include a speech signal from a human being. It may also include other forms of utterances generated by the human speech system. The voice activity detector unit may be adapted to classify a current acoustic environment of the user as a VOICE or NO-VOICE environment.

The hearing aid may comprise an own voice detector for estimating whether or not a given input sound originates from the voice of the user of the system. A microphone system of the hearing aid may be adapted to be able to differentiate between a user's own voice and another person's voice and possibly from NON-voice sounds.

The hearing aid may comprise an acoustic feedback control or echo-cancelling system. Adaptive feedback cancellation has the ability to track feedback path changes over time. It is typically based on a linear time invariant filter to estimate the feedback path, but its filter weights are updated over time. The filter update may be calculated using stochastic gradient algorithms, including some form of the Least Mean Square (LMS) or the Normalized LMS (NLMS) algorithms. They both have the property to minimize the error signal in the mean square sense with the NLMS additionally normalizing the filter update with respect to the squared Euclidean norm of some reference signal.

The hearing aid may further comprise other relevant functionality for the application in question, e.g. compression, noise reduction, etc.

The hearing aid may comprise a hearing instrument, e.g. a hearing instrument adapted for being located at the ear or fully or partially in the ear canal of a user.

In the present context, a hearing aid, e.g. a hearing instrument, refers to a device, which is adapted to improve, augment and/or protect the hearing capability of a user by receiving acoustic signals from the user's surroundings, generating corresponding audio signals, possibly modifying the audio signals and providing the possibly modified audio signals as audible signals to at least one of the user's ears. Such audible signals may e.g. be provided in the form of acoustic signals radiated into the user's outer ears and/or acoustic signals transferred as mechanical vibrations to the user's inner ears through the bone structure of the user's head and/or through parts of the middle ear.

The hearing aid may be configured to be worn in any known way, e.g. as a unit arranged behind the ear with a tube leading radiated acoustic signals into the ear canal or with an output transducer, e.g. a loudspeaker, arranged close to or in the ear canal, as a unit entirely or partly arranged in the pinna and/or in the ear canal, as a unit, e.g. a vibrator, attached to a fixture implanted into the skull bone, etc. The hearing aid may comprise a single unit or several units communicating with each other. The loudspeaker may be arranged in a housing together with other components of the hearing aid or may be an external unit in itself.

A hearing aid may be adapted to a particular user's needs, e.g. a hearing impairment. A configurable signal processing circuit of the hearing aid may be adapted to apply a frequency and level dependent compressive amplification of an input signal. A customized frequency and level dependent gain may be determined in a fitting process by a fitting system based on a user's hearing data, e.g. an audiogram, using a fitting rationale. The frequency and level dependent gain may e.g. be embodied in processing parameters, e.g. uploaded to the hearing aid via an interface to a programming device and used by a processing algorithm executed by the configurable signal processing circuit of the hearing aid.

### BRIEF DESCRIPTION OF DRAWINGS

The aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 schematically shows an exemplary block diagram of a hearing aid according to the present disclosure; and

FIG. 2 shows an exemplary block diagram of a processor according to the present disclosure.

The figures are schematic and simplified for clarity, and they just show details which are essential to the understanding of the disclosure, while other details are left out. Throughout, the same reference signs are used for identical or corresponding parts.

Further scope of applicability of the present disclosure will become apparent from the detailed description given hereinafter. However, it should be understood that the detailed description and specific examples, while indicating preferred embodiments of the disclosure, are given by way of illustration only. Other embodiments may become apparent to those skilled in the art from the following detailed description.

### DETAILED DESCRIPTION OF EMBODIMENTS

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the apparatus and methods are described by various blocks, functional units, modules, components, circuits, steps, processes, algorithms, etc. (collectively referred to as “elements”). Depending upon application, design constraints or other reasons, these elements may be implemented using electronic hardware, computer program, or any combination thereof.

The electronic hardware may include micro-electronic-mechanical systems (MEMS), integrated circuits (e.g. application specific), microprocessors, microcontrollers, digital signal processors (DSPs), field programmable gate arrays (FPGAs), programmable logic devices (PLDs), gated logic, discrete hardware circuits, printed circuit boards (PCB) (e.g. flexible PCBs), and other suitable hardware configured to perform the various functionality described throughout this disclosure, e.g. sensors, e.g. for sensing and/or registering physical properties of the environment, the device, the user, etc. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

The present disclosure presents a hearing aid using one or more biological parameters as a proxy for how much effort is currently needed to listen and to inform a noise reduction algorithm about the needs of the user. The one or more biological parameters may comprise a continuous heart rate measured from in-ear hearing aid sensors such as a sensor for measuring a PPG. The one or more biological parameters may comprise sensor measurement made by a wearable device differing from hearing aid, such as a wrist-born device. The one or more biological parameters may comprise a continuous heart rate measured from an inward turning microphone of the hearing aid.

If a PPG measurement is made it may be carried out by in-ear diodes comprised by the hearing aid. The hearing aid may sample the PPG signal from the ear canal or behind the ear and extract peak-to-peak PPG times as an indicator of instantaneous HR. If the one or more biological parameters are measured by an external device the one or more biological parameters may be transferred via Bluetooth to an accompanying smartphone, which then relays the information to the hearing aid for further analysis.

The processor of the hearing aid may compute the correlation and direction of causality between changes in one or more biological parameters and fluctuations in estimated ambient audio parameters such as SPL and SNR. The one or more audio parameters may be determined by estimators forming part of the processing carried out the hearing aid. The correlation of one or more biological parameters and one or more audio parameters may be quantified by estimating a correlation coefficient (p) continuously across overlapping time-windows that can vary in duration. The correlation between a biological parameter and an audio parameter may be expressed by the covariance of the two parameters divided by the product of their standard deviations.

The threshold for when an estimated correlation is considered different from 0, e.g., negative or positively correlated, may be learned from data, e.g. data in the form of feedback from the user of hearing aid on whether the current processing suits or does not suit the needs of the user.

The time-window selected for the above computation may depend on the application. Longer time-scales, such as hours, may be used for daily assessment, while shorter time-windows, such as 5-10 minutes, may be used for adaptive corrections to ongoing changes in the sound scene or the physiological state of the hearing aid user.

In some embodiments, setting of one or more processing parameters are only carried out if both a correlation measure and a causality measure is present. An example of a causality measure is a direction of causality which may be estimated by a Granger Causality Directionality Index as shown in Porta, Alberto, et al. “Cardiovascular control and time domain Granger causality: insights from selective autonomic blockade.” Philosophical Transactions of the Royal Society A: Mathematical, Physical and Engineering Sciences 371.1997 (2013): 20120161. (DI, Porta et al., 2013). Granger Causality tests if future values of a time-series can be better predicted by a combination of past values of itself and other time-series than by its past values alone. The statistical test is traditionally based on a Wald test of the differences in explained variance between two predictive regression models; one that only contains past values up to a selected lag, and one that also includes past values of another time series. Direction of causality between two timeseries X and Y is then inferred from testing the two hypotheses: H01: X granger cause Y, or H02: Y granger cause X. If both H01 and H02 is accepted, then there exists no linear causal relationship between X and Y. If H01 is accepted but H02 is rejected, then there exists a linear causality running unidirectionally from Y to X. If H01 is rejected but H01 is accepted, then there exists a linear causality running unidirectionally from X to Y. Finally, if both H01 and H02 are rejected, then there is mutual Granger causality between X and Y indicating either a feedback relationship or that changes in X and Y are driven by a common latent variable. To compute a continuous measure of Granger Causality the assessment can be based on a direct comparison between F values assessed over opposite causal directions. Accordingly, the directionality index (DI) may be defined as:

$${DI}_{X \to Y} = {F}_{X \to Y} - {F}_{Y \to X}$$

where FX→Y and FY→X represent the F values assessed from X to Y and vice versa, respectively. DIX→Y>0 indicates that the causal direction from X to Y is prevalent over the reverse one, while DIY→X<0 points out the opposite situation. DI close to 0 might indicate: (i) a full uncoupling between X and Y; (ii) closed-loop interactions between X and Y with none of the causal directions taking real pre-eminence; and (iii) synchronization between X and Y. The estimates of correlation together with the evidence for causality may then support a change in a processing parameter the hearing aid.

To increase the precision of the hearing aid setting processing parameter, physical activity levels may be estimated from activity sensors. This will help determine if high values of ρ occur because ongoing physical activity is linked with specific changes in SPL and SNR, e.g., elevated levels of exercise with consistently high SPL. Activity levels may be estimated by the vector sum of an accelerometer comprised by the hearing aid or as meters/see estimated from an accompanying smartphone GPS. If the activity levels are high, e.g., above a threshold learned from data, together with a low causality directionality index, then no intervention may be made to the processing parameter of the hearing aid.

The thresholds for ρ to indicate if the value is 0 or +High or −High (see Table 1) can be decided by learning from data from individual users. After a period of use, the thresholds are set so that the setting of processing parameter leads to changes in the one or more biological parameters towards a mean of the user.

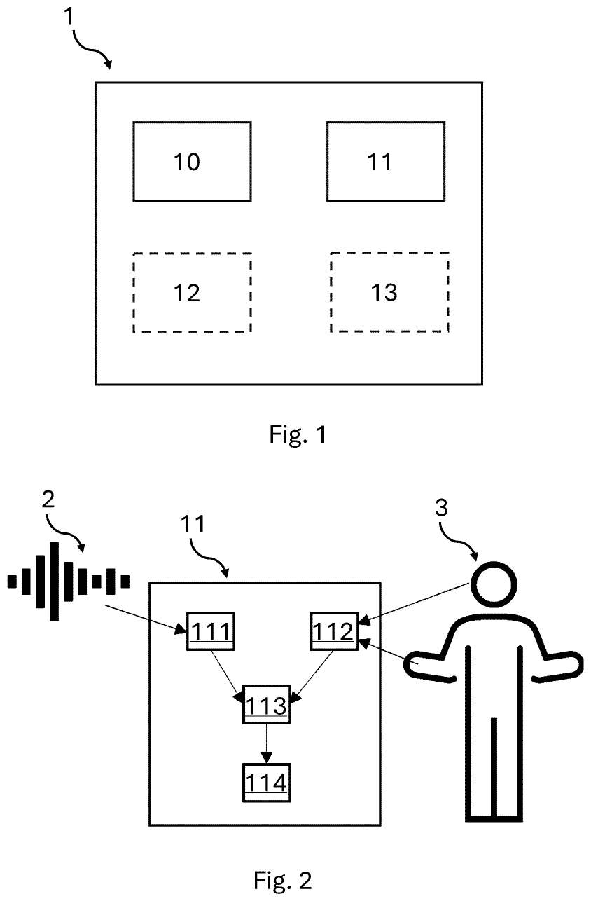

FIG. 1 shows an exemplary block diagram of a hearing aid 1 according to the present disclosure. The hearing aid 1 is configured to be worn by a user 2. The hearing aid 1 comprises an input unit 10 for providing an input audio signal indicative of a surrounding of the hearing aid. The hearing aid 1 comprises a processor 11. The processor is configured to obtain a hearing aid audio signal based on the input audio signal. The processor 11 is configured to determine a first audio parameter based on the input audio signal. The processor 11 is configured to obtain a first sensor signal indicative of a first biological parameter of the user. The processor 11 is configured to determine the first biological parameter based on the first sensor signal. The processor 11 is configured to determine a first correlation between the first audio parameter and the first biological parameter. The processor 11 is configured to set a processing parameter of the hearing aid 1 based on the first correlation. The hearing aid 1 may comprise a first biological sensor 12 configured to determine the first sensor signal. The first biological sensor 12 may comprise an optical sensor, and/or an inward facing microphone. If the first biological sensor 12 comprises a microphone, the microphone may be comprised by the input unit 10 of the hearing aid. The processor 11 may be configured to determine the first correlation continuously over a plurality of time-windows. The first biological parameter may comprise a heart rate of the user. The first audio parameter may comprise a sound pressure level. The processor 11 may be configured to set an aggressiveness of a noise reduction algorithm of the hearing aid 1 based on the first correlation. The processor 11 may be configured to compare the first correlation to a first threshold range. The processor 11 may be configured to, if the first correlation exceeds the first threshold range, increase an aggressiveness of a noise reduction algorithm of the hearing aid 1. The processor 11 may be configured to determine a first estimate of causality between the first audio parameter and the first biological parameter. The processor 11 may be configured to set the processing parameter of the hearing aid 1 based on the first estimate of causality. The first estimate of causality may be a measure of Granger causality. The processor 11 may be configured to obtain a second sensor signal indicative of an activity level of the user. The processor 11 may be configured to determine the activity level based on the second sensor signal. The processor 11 may be configured to set the processing parameter of the hearing aid 1 based on the activity level. The hearing aid may comprise a second sensor 13 configured to determine the second sensor signal. The processor 11 may be configured to, over a period of time, store a plurality of first biological parameters. The processor 11 may be configured to determine a user characteristic based on the plurality of first biological parameter. The processor 11 may be configured to set the processing parameter based on the user characteristic. The processor 11 may be configured to determine a second audio parameter based on the input audio signal. The processor 11 may be configured to determine a second correlation between the second audio parameter and the first biological parameter. The processor 11 may be configured to set the processing parameter of the hearing aid based on the second correlation. The second audio parameter may comprise a signal to noise ratio. The processor 11 may be configured to compare the first correlation to a first threshold range. The processor 11 may be configured to compare the second correlation to a second threshold range. The processor 11 may be configured to, if the first correlation is within the first threshold range or falls short of the first threshold range, and the second correlation is within the second threshold range or falls short of the second threshold range, decrease an aggressiveness of a noise reduction algorithm of the hearing aid 1. The processor 11 may be configured to compare the first correlation to a first threshold range. The processor 11 may be configured to compare the second correlation to a second threshold range. The processor 11 may be configured to, if the first correlation is within the first threshold range or falls short of the first threshold range and the second correlation is within the second threshold range or exceeds the second threshold range except if both the first correlation and the second correlation is within the first threshold range and the second threshold range, maintain the processing parameter.

FIG. 2 shows an exemplary block diagram of a processor 11 according to the present disclosure. The processor 11 is configured to receive an input audio signal 2 indicative of a surrounding of a hearing aid 1 worn by the user 3. From the received input audio signal 2 the processor 11 is configured to determine 111 a first audio parameter. The first audio parameter may be a SPL of the input audio signal. The input audio signal 2 may be obtained via an input unit 10 communicatively connected to the processor 11. The processor is configured to receive a first sensor signal. From the received first sensor signal 2 the processor 11 is configured to determine 112 a first biological parameter of the hearing aid user 3. The first biological parameter may be a HR of the hearing aid user 3. The first sensor signal may be received from a sensor comprised by the hearing aid 1, or from a sensor comprised by a wearable device worn by the hearing aid user 3. From the first audio parameter and the first biological parameter the processor 11 is configured to determine 113 a first correlation. From the determined first correlation the processor 11 is configured to set 114 a processing parameter of the hearing aid 1.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e. to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element, but an intervening element may also be present, unless expressly stated otherwise. Furthermore, “connected” or “coupled” as used herein may include wirelessly connected or coupled. As used herein, the term “and/or” includes any and all combinations of one or more of the associated listed items. The steps of any disclosed method are not limited to the exact order stated herein, unless expressly stated otherwise.

It should be appreciated that reference throughout this specification to “one embodiment” or “an embodiment” or “an aspect” or features included as “may” means that a particular feature, structure, or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art.

The claims are not intended to be limited to the aspects shown herein but are to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. A hearing aid for being worn by a user, comprising:
an input unit for providing an input audio signal indicative of a surrounding of the hearing aid; and
a processor configured to:
obtain a hearing aid audio signal based on the input audio signal,
determine a first audio parameter based on the input audio signal,
obtain a first sensor signal indicative of a first biological parameter of the user,
determine the first biological parameter based on the first sensor signal,
determine a first correlation between the first audio parameter and the first biological parameter, and
set a processing parameter of the hearing aid based on the first correlation.

2. A hearing aid according to claim 1, comprising a first biological sensor configured to determine the first sensor signal.

3. A hearing aid according to claim 2, wherein the first biological sensor comprises an optical sensor, and/or an inward facing microphone facing towards an eardrum of the user.

4. A hearing aid according to claim 1, wherein the first audio parameter comprises a sound pressure level.

5. A hearing aid according to claim 1, wherein the processor is configured to:
set an aggressiveness of a noise reduction algorithm of the hearing aid based on the first correlation.

6. A hearing aid according to claim 1, wherein the processor is configured to:
determine a first estimate of causality between the first audio parameter and the first biological parameter, and
set the processing parameter of the hearing aid based on the first estimate of causality.

7. A hearing aid according to claim 6, wherein the first estimate of causality is a measure of Granger causality.

8. A hearing aid according to claim 1, wherein the processor is configured to:
obtain a second sensor signal indicative of an activity level of the user,
determine the activity level based on the second sensor signal, and
set the processing parameter of the hearing aid based on the activity level.

9. A hearing aid according to claim 8, comprising a second sensor configured to determine the second sensor signal.

10. A hearing aid according to claim 1, wherein the processor is configured to:
over a period of time, store a plurality of the first biological parameters,
determine a user characteristic based on the plurality of first biological parameter, and
set the processing parameter based on the user characteristic.

11. A hearing aid according to according to claim 1, wherein the processor is configured to:
determine a second audio parameter based on the input audio signal,
determine a second correlation between the second audio parameter and the first biological parameter, and
set the processing parameter of the hearing aid based on the second correlation.

12. A hearing aid according to claim 11, wherein the second audio parameter comprises a signal to noise ratio.

13. A hearing aid according to claim 11, wherein the processor is configured to:
compare the first correlation to a first threshold range,
compare the second correlation to a second threshold range, and
if the first correlation is within the first threshold range or falls short of the first threshold range, and the second correlation is within the second threshold range or falls short of the second threshold range, decrease an aggressiveness of a noise reduction algorithm of the hearing aid.

14. A hearing aid according to claim 11, wherein the processor is configured to:
compare the first correlation to a first threshold range,
compare the second correlation to a second threshold range, and
if the first correlation is within the first threshold range or falls short of the first threshold range, and the second correlation is within the second threshold range or exceeds the second threshold range except if both the first correlation and the second correlation is within the first threshold range and the second threshold range, maintain the processing parameter.

15. A hearing aid according to claim 14, wherein the second audio parameter comprises a signal to noise ratio, and wherein the first audio parameter comprises a sound pressure level.
